(19) United States
(12) Patent Application Publication
(10) Pub. No.: US 2005/0129773 A1
(43) Pub. Date: Jun. 16, 2005

(54) Isotretinoin nanoparticulate compositions

(76) Inventors: Inderdeep Bhatia, New Delhi (IN); Rajiv Malik, Wien (AT)

Correspondence Address: Jayadeep R Deshmukh;Ranbaxy Inc, Suite 2100, 600 College Road East, Princeton, NJ, 08540, US

(21) Appl. No.: 10/497,752

(22) PCT Filed: Dec. 6, 2001
(86) PCT No.: PCT/IB01/02329

(51) Int. Cl. A61K 31/203; A61K 9/14

(52) U.S. Cl. 424/489; 514/559

(57) ABSTRACT

The present invention relates to the preparation of a nanoparticulate isotretinoin composition having enhanced bioavailability.

### FIELD OF THE INVENTION

The present invention relates to the preparation of a nanoparticulate isotretinoin composition having enhanced bioavailability.

### BACKGROUND OF THE INVENTION

Isotretinoin (13-cis Vitamin A) is the drug of choice for the treatment of severe, recalcitrant cystic acne and is presently marketed by Hoffman La Roche as Accutane® which is a suspension of isotretinoin filled in soft gelatin capsules.

U.S. Pat. No. 4,464,394 discloses compositions and methods of using 13-cis vitamin A acid against the development of epithelial carcinomas of the skin, gastrointestinal tract, respiratory tract and genito-urinary tract. Although this patent describes pharmaceutical compositions, no data on the bioavailability of the active ingredient is disclosed.

WO 00/25772 discloses that the currently marketed “Accutane®’ formulation of isotretinoin has a mean particle size of 100 μm and has a bioavailability of only about 20%. In this patent application the inventors describe a process for the micronization of isotretinoin to a mean particle size in the range from about 5 μm to about 30 μm. However, no data is presented to show the effect, if any, of this particle size reduction on the bioavailability of isotretinoin.

Isotretinoin is a relatively water insoluble compound which degrades when exposed to light and atmospheric oxygen. It is also highly teratogenic and is contra-indicated in pregnant and lactating women. Due to the low. bioavailability of isotretinoin, higher doses have to be administered. It would therefore be highly desirable to develop dosage forms which are more bioavailable, and consequently the adverse side effects are reduced.

In another PCT application WO 99/52504, a process for the manufacture of (sub)micron sized particles by dissolving in compressed gas and surfactants is described. Isotretinoin is listed as one of the several drugs whose particle size can be reduced by the process disclosed in this application. No specific data on the critical particle size or its effect on the bioavailability has, however, been given in this application. Of course, if the dramatic increase in bioavailability as a function of its particle size was known, the specifics of the critical particle sizes and their effect on the increase in bioavailability would likely have been described.

European Patent Number, EP 184942 discloses pharmaceutical compositions having not more than 22% wax content which according to the inventors is critical in determining the bioavailability. Higher wax content tends to diminish the bioavailability. The median particle size of the drug is also reduced to about 12 μm with a decade particle ratio of less than 25 μm. Said objective of enhanced bioavailability is achieved by controlling both the particle size of isotretinoin and the wax content in the formulation. An increase in the bioavailability of only about 1.6-1.9 times that of commercially available ‘Accutane®’ formulation was reported in this patent.

We have, through extensive experimentation, found that the particle size of isotretinoin used in formulating the final dosage form is critical for determining its bioavailability.

### SUMMARY OF THE INVENTION

The present invention provides a pharmaceutical composition of isotretinoin comprising isotretinoin having a mean particle size (d50) of less than about 1000 nm (1 μm).

The present invention also provides a process for the reduction of the particle size of isotretinoin to less than about 1000 nm (1 μm), without any loss in potency.

The present invention further provides a method of using isotretinoin having a mean particle size (d50) of less than about 1000 nm (1 μm) for the treatment of severe recalcitrant cystic acne.

Finally, the present invention provides a pharmaceutical composition comprising isotretinoin wherein the AUC and Cmax values obtained by the composition were at least three times increased as compared to the commercially available formulation of isotretinoin sold under the tradename of Accutane®

The production of isotretinoin with reduced particle size and thereby increased bioavailability would have obvious benefits of achieving the desired therapeutic effects by the administration of lower amounts of drug.

It is known that conventional techniques of micronization such as hammer mill, ball mill or air attrition mill result in a significant loss in potency of isotretinoin as it is very sensitive to oxidation. It is also known that isotretinoin powder can be micronized with negligible loss in potency by suspending it in an oily vehicle and milling it. Heretofore, the particle size reduction only to particle size between 5 μm-30 μm has been reported by this wet milling method.

In “The Theory and Practice of Industrial Pharmacy”, Lachman et al note that although wet milling is beneficial for reducing the particle size to about 10μ(10,000 nm), flocculation prevents any further reduction in the particle size by this method. Further reduction of the particle size to sub (micron) levels often requires highly sophisticated techniques that require building up often expensive and specialized infrastructure. Surprisingly, we have found that it is possible to reduce the particle size of isotretinoin to submicron levels by utilizing the simple process of wet milling.

Therefore, in accordance with the present invention, isotretinoin obtained in the conventional coarse form preferably having a particle size of less than about 250 μm is mixed with a carrier to form a medicated suspension. Isotretinoin may be present from a concentration of about 0.1 gm to 60 gm per 100 ml of the medicated suspension. Preferably it is present from about 5-30 gm per 100 ml of the medicated suspension.

The medicated suspension is then subjected to impact, shear and cavitation forces produced by high sheer homogenization or wet milling to get nanoparticulate drug having a mean particle size (d50) of less than about 1000 nm. Particle size reduction is preferably carried out using wet mills. Examples of such mills include Dispermat SL (VMA_Getzmann GMBH, Germany) and bead mills such as the Dyno-Mill Type KDL (Willy Bachofen AG, Maschinenfabrik, Switzerland).

Bead mills such as the Dyno millwork on the principle of transmitting the energy for dispersion and grinding to the grinding beads via an early exchangeable agitator shaft. While processing, the material to be milled is constantly fed into the mill by a separate product pump. The grinding media for particle size reduction can be selected from spherical or particulate rigid media, less than about 3 mm in diameter and more preferably less than about 1 mm in diameter. The selection of material for the grinding media is not believed to be critical to our process. We have found that zirconium oxide such as 95% ZrO stabilized with magnesia, zirconium silicate and glass grinding media provide good particle size reduction. However, other media such as stainless steel, titania, alumina and 95% ZrO stabilized with yttrium are also expected to be useful.

The time taken for the reduction in particle size depends upon the mechanical means and the processing conditions selected. Processing times of less than one day have provided the desired results in high sheer milling such as the dyano mill. The entire process is preferably carried out under controlled ambient temperature conditions.

The mean particle size (d50) is reduced to less than about 1000 nm. Preferably, the d50 is less than about 800 nm. More preferably, the d50 is less than about 500 nm. The d90 of this isotretinoin is less than about 4000 nm. Preferably, the d90 is less than about 3000 nm. More preferably, the d90 is less than about 1500 nm. Analysis of the particle size of isotretinoin is carried out using any conventional particle size analyzer (e.g. the Malvern Master Sizer™).

The nanonized isotretinoin thus obtained showed no loss in potency as a result of particle size reduction. The isotretinoin composition thus produced was storage stable.

The carrier material used to form the medicated suspension is selected from the group consisting of peanut oil, fractionated coconut oil marketed under the trade name of “Miglyol®” soyabean oil, sesame oil, mineral oil, cotton seed oil, polyethylene glycol, and mixtures thereof.

Subsequent to the reduction in the particle size, the nanoparticulate isotretinoin-carrier is mixed with a suspending agent, and optionally with other pharmaceutically acceptable excipients, before being encapsulated into a soft gelatin capsule dosage form.

The suspending agent used in accordance with the present invention is a standard wax mixture as described by J P Stanley, in The Theory and Practice of Industrial Pharmacy, L. Lackman, H. A. Lieberman and J. L. Klanig, eds; 2nd ed; Lea & Febiger, Phila (1976) comprising 1 part hydrogenated soyabean oil, 1.2 parts white wax and 4.2 parts hydrogenated vegetable oil. In addition to the standard wax mixture, beeswax, paraffin wax and glyceryl monostearate may also be employed. The suspending agent may be present in amounts from about 30% to about 40%, by weight of the formulation.

The formulation of the present invention may further contain other suitable pharmaceutical excipients such as antioxidants, chelating agents and surfactants.

The anti-oxidant employed in the present invention may be selected from the group consisting of α-tocopherol, butylated hydroxy anisole (BHA), butylated hydroxytoluene (BHT), ascorbyl palmitate and propyl gallate. Chelating agents may be chosen from amongst those conventionally known in the art such as disodium edetate and calcium disodium edetate.

Suitable surfactants which can be employed include lecithin, sorbitan monostearate, polysorbates prepared from lauric, palmitic, stearic and oleic acid; mononylphenyl ethers of polyethyleneglycols such as nanoxynols; polyoxyethylene monoesters such as polyoxyethylethylene monostearate, polyoxyethylene, monolaurate, polyoxyethylene monooleate, dioctyl sodium sulfosuccinate, sodium lauryl sulfate and poloxamers.

### DETAILED DESCRIPTION OF THE INVENTION

The present invention is illustrated by, but not limited to, the following examples:

### EXAMPLE 1

A nanoparticulate dispersion of isotretinoin was prepared using the DYNO mill (model KDL, manufactured by Willy A Bachoffen AG Maschinefabrik). 250 g Isotretinoin was mixed with 1500 ml soya oil and homogenized. The mill grinding chamber was partially (80%) filled with zirconium silicate beads and the medicated suspension was continuously circulated through the media mill at an agitator disc speed of 10 m/sec for one hour at a product feed rate of 10-15 kg per hour. The final dispersion was analyzed for particle size reduction and the values obtained are given in Table 1.

TABLE 1
Representative particle size data

| | | | | | (d90) 90^{th} | |
|---|---|---|---|---|---|---|
| | (d10)10^{th} percentile | | (d50)50^{th} percentile | | percentile | |
| particle size | 0.061 | μm | 0.113 | μm | 0.587 | μm |
| | (61 | nm) | (113 | nm) | (587 | nm) |

Investigations were conducted in order to determine the effect of particle size on the bioavailability of isotretinoin in the formulations of the invention. The blood levels of the drug were compared with that of the commercially available formulation sold as “Accutane™”.

The mean particle size of isotretinoin was reduced to d50 values of 100 μm, 3.5 μm, 25.7 μm and 0.395 μm (395 nm) The isotretinoin of different particle sizes was formulated as described in Table 2 and encapsulated in a soft gelatin capsule.

TABLE 2
Isotretinoin Formulation

| Ingredient | mg per capsule |
|---|---|
| Isotretinoin | 40.0 |
| Hydrogeanted Soyabean oil | 15.30 |
| Hydrogenated Vegetable oil | 64.26 |
| White wax | 18.36 |
| Edetate disodium | 0.16 |
| Butylated hydroxyanisole | 0.032 |
| Soyabean oil | 180.0 |
| Total | 320.00 |

Nanoparticulate isotretinoin was prepared as described in Example 1. The hydrogenated soyabean oil, white wax and hydrogenated vegetable oil were melted in a separate vessel and mixed with the milled isotretinoin mixture, edetate disodium and butylated hydroxyanisole. The resulting blend was filled into soft gelatin capsules.

Pharmacokinetic Data

The effect of particle size on the bioavailability of isotretinoin was studied and compared with that of the commercially available formulation of isotretinoin sold under the brand name “Accutane®”. The area under the plasma concentration-time curve (AUC) and the maximum concentration (Cmax) for orally administered isotretinoin of different particle sizes formulated as soft gelatin capsules was compared.

As isotretinoin behaves in a dose linear fashion, the Pk data from the above studies were dose normalized to the same dosage strength of 22.5 mg for all particle sizes and compared (Table 3).

TABLE 3
Comparison of the AUC and Cmax values of different
particle sizes of isotretinoin. Dose normalised to 22.5 mg

| Mean Particle size | Cmax | | AUC 0-α |
|---|---|---|---|
| (d50) | (ng/ml) | AUC0-t(ng · h/ml) | (ng · h/ml) |
| Accutane capsules | 113.80 | 1106.97 | 1180.4 |
| Isotretinoin capsules | 105.74 | 1059.75 | 1135.67 |
| (113 μm) | | | |
| Isotretinoin capsules | 202.38 | 2081.04 | 2125.17 |
| (25.7 μm) | | | |
| Isotretinoin capsules | 388.56 | 4007.56 | 4367.60 |
| (0.395 μm) | | | |

The commercially marketed Accutane® formulation and our isotretinoin capsules having a mean particle size (d50) of about 100 μm had similar AUC and Cmax values. Reduction of particle size to 25.7 μm increased the bioavailability by about 1.9 times that of Accutane®. On further reduction of the particle size to the nanoparticulate range (about 0.395 μm), there was a substantial increase in the bioavailability of isotretinoin to more than 3.5 times that of Accutane®.

While the invention has been described by reference to specific examples, this was for purposes of illustration only. Numerous alternative embodiments will be apparent to those skilled in the art and are considered to be within the scope of the invention.

1. A pharmaceutical composition comprising isotretinoin having a mean particle size (d50) of less than about 1000 nm (1 μm).

2. The composition according to claim 1 wherein the mean particle size (d50)of isotretinoin is preferably less than about 800 nm.

3. The composition according to claim 1 wherein the mean particle size (d50) of isotretinoin is more preferably less than about 500 nm.

4. The composition according to claim 1 wherein the d90 value is less than about 4000 nm.

5. The composition according to claim 1 wherein the d90 value is preferably less than about 3000 nm.

6. The composition according to claim 1 wherein the d90 value is more preferably less than about 1500 nm.

7. A process for the manufacture of nanonised isotretinoin composition wherein coarse isotretinoin is suspended in an oily or other pharmaceutically acceptable carrier to form a medicated suspension and subjected to mechanical means to reduce the mean particle size (d50) to less than about 1000 nm (1 μm).

8. The process according to claim 7 wherein the mean particle size (d50) is preferably reduced to less than about 800 nm.

9. The process according to claim 7 wherein the mean particle size (d50) is more preferably reduced to less than about 500 nm.

10. The process according to claim 7 wherein the oily carrier comprises soyabean oil, peanut oil, fractionated coconut oil, mineral oil, cotton seed oil, polyethylene glycol, and mixtures thereof.

11. The process according to claim 7 wherein the mechanical means used is bead milling.

12. A pharmaceutical composition in a unit oral dosage form comprising a therapeutically effective amount of isotretinoin having a mean particle size (d50) of less than about 1000 nm (1 μm) and a carrier.

13. The pharmaceutical composition according to claim 12 wherein the carrier is selected from the group consisting of peanut oil, fractionated coconut oil, soyabean oil, sesame oil, mineral oil, cotton seed oil, polyethylene glycol, and mixtures thereof.

14. The pharmaceutical composition of claim 12 further comprises a suspending agent, and optionally other pharmaceutically acceptable excipents.

15. The pharmaceutical composition according to claim 14 wherein the suspending agent is a wax mixture comprising 1 part hydrogenated soyabean oil, 1.2 parts white wax and 4.2 parts hydrogenated vegetable oil.

16. The pharmaceutical composition of claim 15 wherein the suspending agent additionally contains beeswax, paraffin wax, glyceryl monostearate, and mixtures thereof.

17. The pharmaceutical composition according to claim 14 wherein the suspending agent comprises about 30% to about 40% by weight of the formulation.

18. The pharmaceutical composition according to claim 14 wherein the pharmaceutically acceptable excipients are chelating agents, antioxidants and surfactants.

19. The pharmaceutical composition according to claim 18 wherein the chelating agent is selected from amongst disodium edetate and calcium disodium edetate.

20. The pharmaceutical composition according to claim 18 wherein the antioxidant is selected from the group consisting of α-tocopherols, butylated hydroxyanisole, butylated hydroxytoluene, ascorbyl palmitate and propyl gallate.

21. The pharmaceutical composition according to claim 18 wherein the surfactant is selected from the group consisting of lecithin, sorbitan monostearate, polysorbates, monononyl ethers of polyethylene glycols, polyoxyethylene monostearate, polyoxyethylene monolaurate, diocyl sodium succinate, sodium lauryl sulfate and poloxamers.

22. The pharmaceutical composition as described in claim 1, for use in the treatment of severe, recalcitrant cystic acne.

23. A pharmaceutical composition comprising isotretinoin wherein the AUC and Cmax values obtained by the composition are at least three times increased as compared to the commercially available formulation sold under the tradename of Accutane®.

24. The pharmaceutical composition according to claim 23 wherein the mean particle size (d50) of isotretinoin is less than 1000 nm.

25. The pharmaceutical composition according to claim 23 wherein the mean particle size (d50) of isotretinoin is less than 800-nm.

26. The pharmaceutical composition according to claim 23 wherein the mean particle size (d50) of isotretinoin is less than 500 nm.
